(19) United States
(12) Patent Application Publication
(10) Pub. No.: US 2003/0129231 A1
(43) Pub. Date: Jul. 10, 2003

(54) Sustained release oxycodone formulations with no fed/fast effect

(76) Inventors: Benjamin Oshlack, New York, NY (US); Mark Chasin, Manalapan, NJ (US)

Correspondence Address: DAVIDSON, DAVIDSON & KAPPEL, LLC, 14th Floor, 485 Seventh Avenue, New York, NY, 10018, US

(21) Appl. No.: 10/272,340

(22) Filed: Oct. 16, 2002

Related U.S. Application Data
(63) Continuation of application No. 10/066,884, filed on Feb. 4, 2002, now abandoned.
Application No. 10/066,884 is a continuation of application No. 09/826,441, filed on Apr. 5, 2001, now abandoned.
Application No. 09/826,441 is a continuation of application No. 09/435,010, filed on Nov. 5, 1999, now abandoned.
Application No. 09/435,010 is a continuation of application No. 08/652,645, filed on May 31, 1996, now abandoned.

(51) Int. Cl. A61K 31/485; A61K 9/20; A61K 9/22

(52) U.S. Cl. 424/468; 514/282

(57) ABSTRACT

A solid controlled release, oral dosage form, the dosage form comprising a therapeutically effective amount of oxycodone or a salt thereof together with a sustained release carrier which causes the formulation to preferentially release the drug in low pH (e.g., gastric fluid) is bioavailable and does not exhibit a fed/fast effect.

### BACKGROUND OF THE INVENTION

The present invention relates to solid, controlled release oral dosage forms for use in the treatment of moderate to severe pain, which do not exhibit a altered absorption of active ingredient in the presence of food.

It has been previously known in the art that controlled release oxycodone formulations could be prepared via sustained release coated bead or sustained release matrix formulations. For example, U.S. Pat. No. 5,266,331, assigned to the assignee of the present invention and hereby incorporated by reference in its entirety, teaches controlled release oxycodone formulations prepared utilizing a suitable sustained release matrix. The preparations described in the '331 patent preferably exhibit an in-vitro dissolution rate of the dosage form, when measured by the USP Paddle Method at 100 rpm in 900 ml aqueous buffer (pH between 1.6 and 7.2) at 37° C., which is between 12.5 and 42.5% (by wt) oxycodone released after 1 hour, between 25 and 55% (by wt) released after 2 hours, between 45 and 75% (by wt) released after 4 hours and between 55 and 85% (by wt) released after 6 hours, the in-vitro release rate being independent of pH between pH 1.6 and 7.2 and chosen such that the peak plasma level of oxycodone obtained in vivo occurs between 2 and 4 hours after administration of the dosage form.

It further known that suitable sustained release oxycodone formulations are prepared using sustained release coated spheroid formulations, as described in U.S. Pat. No. 5,508,042 (Oshlack et al.) assigned to the assignee of the present invention and hereby incorporated by reference in its entirety.

There is a need in the art for controlled release formulations that do not exhibit a food effect.

### SUMMARY OF THE INVENTION

The present invention is directed in part to sustained release formulations of oxycodone which do not have a significant fed/fast effect, and methods for the preparation of the same.

The sustained release oxycodone formulations of the present invention comprise an effective amount of oxycodone or a pharmaceutically acceptable salt thereof, and a sustained release carrier which preferentially causes the formulation to release the oxycodone in fluids having a relatively lower (acidic) pH.

The present invention is further directed to the preparation of sustained release oxycodone formulations which do not exhibit a significant fed/fast effect, which is accomplished by utilizing a sustained release carrier which preferentially causes the formulation to release the oxycodone in fluids having a relatively lower (acidic) pH.

The present invention is further directed to a method of treating patients in the need of analgesia with oxycodone in a manner which provides a sustained effect in-vivo, which effect does not significantly vary with respect to the gastric contents of the patient, by utilizing a sustained release carrier which preferentially causes the formulation to release the oxycodone in fluids having a relatively lower (acidic) pH.

In preferred embodiments of the invention, the oxycodone is in the form of the hydrochloride salt, or other pharmaceutically acceptable salts known to those skilled in the art, such as oxycodone terephthalate. The hydrochloride salt is preferred.

In further preferred embodiments of the invention, the sustained release carrier is an acrylic polymer, or other retardants such as cellulose polymers. The sustained release carrier may comprise part of a matrix or may be utilized as a coating on substrate containing the drug e.g., a tablet core or particles in a multi-particulate formulation.

For purposes of the present invention, “pH-dependent” means that the formulation provides a greater release in the amount of oxycodone released at acidic pH, e.g., pH 1.0 found in the human stomach, than the significantly higher pH's found in the intestinal tract, e.g., pH 7.6. More particularly, “pH-dependent”, for purposes of the present invention, means that the sustained release oxycodone formulation includes a sustained release carrier which causes the formulation to possess a dissolution profile (rate of drug substance release) which is essentially insensitive to variations in dissolution media pH in the range of about pH 1.2 to about pH 6.8, and which possess a dissolution rate of drug substance release which decreases as the pH of the dissolution medium becomes basic.

For purposes of the present invention, “independent of pH” means that there is virtually no difference, at any given time, between the amount of oxycodone released at pH 1.6 and the amount released at any other pH up to, and including, pH 7.2 (when measured in vitro using the LSP Paddle Method at 100 rpm in 900 ml aqueous buffer). In other words, the dissolution curves are virtually superimposible. The amounts released being, in all cases, a mean of at least three experiments.

For purposes of the present invention, the phrase “no fed/fast effect” means that there is less than 20% difference between the pharmacokinetic parameters (determined from blood levels of active drug) with respect to the valves for maximum blood plasma concentration (i.e., Cₘₐₓ and area under the curve (i.e., AUC) obtained when patients are dosed with the formulation on an empty stomach as compared to when the drug formulation is administered to patients who have ingested a high-fat meal, as defined by the U.S. Food and Drug Administration or corresponding foreign regulatory body (i.e., the “fed state”) and a food effect is considered to exist, where these differences are greater than 20%.

The term “sustained-release” for purposes of the present invention means that the oral dosage form provides a release of the oxycodone contained therein over a period from about 8 to about 24 hours or more. The sustained-release formulations of the present invention preferably release the drug (e.g., oxycodone) at such a rate that blood (e.g., plasma) levels are maintained within the therapeutic range but below toxic levels over a period of time greater than 8 hours, more preferably for about 12 to about 24 hours, or longer.

The term “bioavailable” is defined for purposes of the present invention as the total amount of a drug substance that is absorbed is considered to be substantially equivalent as compared to the immediate release dosage form, to provide the desired therapeutic effect after administration of a unit dosage form. Generally, the bioavailability of a given dosage form is determined by comparison to a known reference drug product, as commonly determined and accepted by Governmental Regulatory Agencies, such as the United States FDA.

### BRIEF DESCRIPTION OF THE DRAWINGS

The following drawings are illustrative of embodiments of the invention and are not meant to limit the scope of the invention as encompassed by the claims:

FIG. 1 is a graphical representation of the plasma drug levels, both in the fed and fasted states, obtained from a comparative formulation which preferentially releases the drug at high pH's.

FIG. 2 is a graphical representation of the rate of drug release of Example 3 in dissolution media of differing pH.

FIG. 3 is a graphical representation of the plasma drug levels, both in the fed and fasted states, obtained from sustained release oxycodone formulations prepared in accordance with the presently claimed invention.

### DETAILED DESCRIPTION

Oxycodone hydrochloride is more soluble in gastric fluid (pH about 1) than in intestinal fluid (pH about 7.5). Currently marketed immediate-release oxycodone hydrochloride formulations (e.g., Roxicodone™ 5 mg tablets, commercially available from Roxane Labs) exhibit an increased solubility of oxycodone in a lower pH environment. Human clinical studies have demonstrated that there is a substantial food effect exhibited by these dosage forms.

Since all of the drug from these known immediate-release dosage forms are released in the low pH of the stomach, one skilled in the art would therefore predict that the best chance to decrease this food effect would be to ensure that most of the oxycodone be released preferentially in a higher pH environment rather than in a low pH environment.

Generally, it is known in the art that certain sustained release formulations may exhibit a “food effect”, i.e., the dosage forms usually exhibit decreased Cₘₐₓ and/or AUC of the drug when administered in the presence of food. In order to avoid such a food effect, it is known in the art that enteric coatings may be employed, which allows the drug to be absorbed after the formulation passes through the (full) stomach. Such formulations do not release significant amounts of active ingredient until the dosage form is in the higher pH environment of the small intestine.

However, when oxycodone formulations are prepared which preferentially release the drug in high pH environments, a substantial food effect is still observed. Such formulations can be prepared, for example, by utilizing enteric coatings. This discovery is in itself not surprising. One skilled in the art might predict that changing the pH dependency, i.e., providing a sustained release oxycodone formulation which preferentially releases the drug in high pH instead of low pH, would only help decrease the food effect.

Surprisingly, however, the inventors of the presently claimed invention have prepared oral sustained release oxycodone formulations which despite the problems set forth above, do not exhibit a significant fed/fast effect. This has been accomplished by preparing the sustained release formulation in such a manner that the oxycodone is released more favorably in low pH (e.g., gastric fluid) rather than high pH (e.g., intestinal fluid).

In one preferred embodiment the sustained-release opioid oral dosage form of the present invention includes from about 2 to about 500 mg oxycodone, and more preferably from about 5 mg to about 400 mg oxycodone, based on the hydrochloride salt.

The sustained release carrier may be incorporated into a matrix with the drug (oxycodone), which matrix may comprise a tablet core or a particle (in a multi-particulate formulation. That matrix may be additionally coated with a sustained release carrier if so desired. Either the sustained release carrier in the matrix, or the sustained release carrier in the coating, or both, must cause the final formulation to provide a pH-dependent dissolution as defined herein, and a sustained release oxycodone formulation which does not exhibit a fed/fast effect, as that term is defined herein.

Alternatively, the oxycodone may be incorporated into a substrate containing the drug in immediate release form, e.g., an immediate release tablet core comprising oxycodone together with pharmaceutically acceptable excipients (inert diluents, binders, etc.), or spheroids comprising the drug together with a pharmaceutically acceptable spheronizing agent (such as microcrystalline cellulose), or inert beads coated with the drug, any of which are thereafter coated with a sustained release carrier such that the final formulation provides a pH-dependent dissolution as defined herein, and a sustained release oxycodone formulation which does not exhibit a fed/fast effect, as that term is defined herein.

The term “multiparticulate” is thus defined as encompassing beads, pellets, and any other multiparticulate systems which may be orally administered.

The sustained release formulation may be prepared for example, in accordance with any of the procedures set forth in U.S. Pat. Nos. 5,266,331; 5,286,493; 5,478,577; 5,273,760; 4,861,598; and 5,508,042, all of which are hereby incorporated by reference herein. Of course, any other methods known to those skilled in the art which may be utilized to prepare the pH-dependent dosage of the present invention which may be utilized to prepare the pH-dependent dosage forms of the present invention may be used and are contemplated to be within the scope of the appended claims.

The sustained-release dosage forms of the present invention generally achieve and maintain therapeutic levels substantially without significant increases in the intensity and/or degree of concurrent side effects, such as nausea, vomiting or drowsiness, which are often associated with high blood levels of opioid analgesics. There is also evidence to suggest that the use of the present dosage forms leads to a reduced risk of drug addiction.

In the present invention, the oral oxycodone formulations have been formulated to provide for an increased duration of analgesic. Surprisingly, these formulations, at comparable daily dosages of conventional immediate-release oxycodone, are associated with a lower incidence in severity of adverse drug reactions and can also be administered at a lower daily dose than conventional oral medication while maintaining pain control.

The sustained-release dosage forms of the present invention may further include one or more additional drugs which may or may not act synergistically with the opioid analgesics of the present invention. Examples of such additional therapeutically active agents include non-steroidal anti-inflammatory agents, including ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid tolfenamic acid, diflurisal, flufenisal, piroxicam, sudoxicam or isoxicam, and the like. Other suitable additional drugs which may be included in the dosage forms of the present invention include acetaminophen, aspirin, salicylate-derived analgesics and antipyretics or salts thereof, and other non-opioid analgesics.

The additional (non-opioid) therapeutically active agent may be included in controlled release form or in immediate release form. The additional drug may be incorporated into the controlled release matrix along with the opioid; incorporated as a separated controlled release layer or immediate release layer; or may be incorporated as a powder, granulation, etc., in a gelatin capsule with the extrudates of the present invention.

The Sustained Release Carrier

The sustained-release formulations of the present invention preferably includes at least one retarding material. The retarding material will preferably impart sustained release of the opioid analgesic to the final formulation. Suitable retarding materials which may be used in accordance with the present invention include alkylcelluloses such as natural or synthetic celluloses derivatives (e.g. ethylcellulose), acrylic and methacrylic acid polymers and copolymers, shellac, zein, wax-type substances including hydrogenated castor oil or hydrogenated vegetable oil, and mixtures thereof. Specifically, the retarding material may comprise natural or synthetic waxes, fatty alcohols (such as lauryl, myristyl, stearyl, cetyl or preferably cetostearyl alcohol), fatty acids, including but not limited to fatty acid esters, fatty acid glycerides (mono-, di-, and tri-glycerides), hydrogenated fats, hydrocarbons, normal waxes, stearic aid and stearyl alcohol. These may be hydrophilic or hydrophobic materials. Mixtures of any of the foregoing, and other pharmaceutically acceptable sustained-release carrier materials known to those skilled in the art may also be used as the retarding material. The final sustained-release oral dosage form may contain up to 60% (by weight) of at least one digestible, long chain hydrocarbon.

In certain preferred embodiments, a combination of two or more retarding materials are included in the sustained-release carrier. Any pharmaceutically acceptable retarding material may be used, with the proviso that the formulation in toto must impart a sustained release of the active agent and preferential release of the drug at low pH may be used in accordance with the present invention.

In certain preferred embodiments of the present invention, the retarding material is a pharmaceutically acceptable acrylic polymer, including but not limited to acrylic acid and methacrylic acid copolymers, methyl methacrylate, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. In other embodiments, the sustained release carrier may further include a relatively hydrophilic material, including but not limited to materials such as hydroxyalkylcelluloses such as hydroxypropylmethylcellulose and mixtures of the foregoing.

A preferred acrylic polymer for use in accordance with the present invention is Eudragit RS30D commercially available from Rohm Pharma.

A pharmaceutically acceptable plasticizer may also be included in the sustained-release carrier or matrix of the present invention. A non-limiting list of plasticizers includes include water insoluble plasticizers such as dibutyl sebacate, diethyl phthalate, triethyl citrate, tibutyl citrate, and triacetin, although it is possible that other water-insoluble plasticizers (such as acetylated monoglycerides, phthalate esters, castor oil, etc.) may be used. Triethyl citrate is an especially preferred plasticizer.

The final formulation may be, e.g. a capsule or a tablet. When the final dosage form is a tablet, the tablet may be coated with a film coat to provide a protective layer. Suitable film coats which may be used include immediate release, cellulose polymers, acrylic resins, pyrolidone derivatives and other immediate release long chain hydrocarbons.

In addition to the above ingredients, a sustained-release carrier or matrix may also contain suitable quantities of pharmaceutical adjuvants, e.g., diluents, lubricants, binders, granulating aids, colorants, flavorants and glidants that are conventional in the pharmaceutical art. A non-limiting list of suitable adjuvants include spray dried lactose, polyvinylpyrrolidone (PVP), talc, magnesium stearate, and mixtures thereof. The quantities of these additional materials will be sufficient to provide the desired effect to the desired formulation. The final formulation may contain up to about 50% by weight of the final dosage form, if desired.

Other examples of pharmaceutically acceptable carriers and excipients that may be used to formulate oral dosage forms are described in the Handbook of Pharmaceutical Excipients, American Pharmaceutical Association (1986), incorporated by reference in its entirety.

In certain preferred embodiments, the sustained release formulations of the present invention may be prepared as a melt-extruded matrix. Incorporation in the matrix may be effected, for example, blending the oxycodone, together with at least one hydrophobic material and preferably a second hydrophobic material to obtain a homogeneous mixture. The homogeneous mixture is then heated to a temperature sufficient to at least soften the mixture sufficiently to extrude the same. The resulting homogeneous mixture is then extruded, e.g., using a twin-screw extruder, to form an extrudate. The extrudate is preferably cooled and cut into multiparticulates by any means known in the art, and are preferably cut to form strands. The extrudates are cooled and cut into multiparticulates. The multiparticulates are then divided into unit doses. The strands preferably have a diameter of from about 0.1 to about 5 mm and provides sustained release of the therapeutically active agent for a time period of from about 8 to about 24 hours. The second hydrophobic material is preferably a hydrophobic material may comprise one or more water-insoluble wax-like thermoplastic substances possibly mixed with one or more wax-like thermoplastic substances being less hydrophobic than said one or more water-insoluble wax-like substances.

Preferably, the retarding materials used in this embodiment have a melting point from about 30 to about 200° C., preferably from about 45 to about 90° C. Also, optionally, the melt-extruded material may include a binder (e.g., vegetable or castor oil, paraffin, higher aliphatic alcohols, higher aliphatic acids, long chain fatty acids, fatty acid esters, normal waxes and/or wax-like substances, and/or mixtures thereof. Suitable waxes include, for example, beeswax, glycowax, castor wax, carnauba wax and the like. For purposes of the present invention, a wax-like substance is defined as any material which is normally solid at room temperature and has a melting point of from about 30 to about 100° C. This embodiment may be manufactured, for example, in accordance with the procedures set forth in U.S. patent application Ser. No. 08/334,209 filed Nov. 4, 1994, and hereby incorporated by reference.

Alternatively, the melt-extruded material is prepared without the inclusion of the therapeutically active agent, which is added thereafter to the extrudate. Such formulations may have the oxycodone blended together with the extruded matrix material, and then the mixture would be tabletted in order to provide a slow release formulation.

An optional process for preparing the melt extrusions, multiparticulates and unit doses of the present invention includes directly metering into an extruder a water-insoluble material, a therapeutically active agent, and an optional hydrophobic material; heating said homogenous mixture; extruding said homogenous mixture to thereby form strands; cooling said strands containing said homogeneous mixture; and cutting said strands into particles having a size from about 0.1 mm to about 12 mm; and dividing said particles into unit doses. In this aspect of the invention, a relatively continuous manufacturing procedure is realized.

The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

The melt extruded multiparticulate system can be, for example, in the form of granules, spheroids or pellets depending upon the extruder exit orifice. For purposes of the present invention, the terms “melt-extruded multiparticulate(s)” and “melt-extruded multiparticulate system(s)” and “melt-extruded particles” shall refer to a plurality of units, preferably within a range of similar size and/or shape and containing one or more active agents and one or more excipients, preferably including a retardant as described herein. In this regard, the melt-extruded multiparticulates will be of a range of from about 0.1 to about 12 mm in length and have a diameter of from about 0.1 to about 5 mm. In addition, it is to be understood that the melt-extruded multiparticulates can be any geometrical shape within this size range such as beads, microspheres, seeds, pellets, etc.

In one preferred embodiment, oral dosage forms are prepared to include an effective amount of melt-extruded multiparticulates within a capsule. For example, a plurality of the melt-extruded multiparticulates may be placed in a gelatin capsule in an amount sufficient to provide an effective sustained-release dose when ingested and contacted by gastric fluid.

In another preferred embodiment, a suitable amount of the multiparticulate extrudate is compressed into an oral tablet using conventional tableting equipment using standard techniques. These multiparticulates can also he screened or melted into a smaller particle size granulation and then tabletted or filled into capsules. Techniques and compositions for making tablets (compressed and molded), capsules (hard and soft gelatin) and pills are also described in Remington's Pharmaceutical Sciences, (Arthur Osol, editor), 1553-1593 (1980), incorporated by reference herein.

In yet another preferred embodiment, the extrudate can be shaped into tablets as set forth in U.S. Pat. No. 4,957,681 (Klimesch, et. al.), described in addtional detail above and hereby incorporated by reference.

Optionally, the sustained-release melt-extruded multiparticulate systems or tablets can be coated, or the gelatin capsule can be further coated, with a sustained-release coating comprising one of the sustained release carriers described above to provide a preferential release of the drug at low pH. Such coatings preferably include a sufficient amount of sustained release carrier to obtain a weight gain level from about 2 to about 30 percent. The solvent which is used for the hydrophobic material in the coating may be any pharmaceutically acceptable solvent, including water, methanol, ethanol, methylene chloride and mixtures thereof.

The unit dosage forms of the present invention may further include combinations of multiparticulates containing one or more of the therapeutically active agents disclosed above before being encapsulated. Furthermore, the unit dosage forms can also include an amount of an immediate release therapeutically active agent for prompt therapeutic effect. The immediate release therapeutically active agent may be incorporated, e.g., as separate pellets within a gelatin capsule, or may be coated on the surface of the compressed tablet which has been prepared from the multiparticulate extrudate as set forth above.

The controlled-release profile of the fomulations of the invention can be altered, for example, by varying the amount of retardant, e.g., hydrophobic polymer, by varying the amount of plasticizer relative to hydrophobic polymer, by the inclusion of additional ingredients or excipients, by altering the method of manufacture, etc.

Typical melt extrusion systems capable of carrying-out the present invention include a suitable extruder drive motor having variable speed and constant torque control, start-stop controls, and ammeter. In addition, the system will include a temperature control console which includes temperature sensors, cooling means and temperature indicators throughout the length of the extruder. In addition, the system will include an extruder such as twin-screw extruder which consists of two counter-rotating intermeshing screws enclosed within a cylinder or barrel having an aperture or die at the exit thereof. The feed materials enter through a feed hopper and is moved through the barrel by the screws and is forced through the die into strands which are thereafter conveyed such as by a continuous movable belt to allow for cooling and being directed to a pelletizer or other suitable device to render the extruded ropes into the multiparticulate system. The pelletizer can consist of rollers, fixed knife, rotating cutter and the like. Suitable instruments and systems are available from distributors such as C.W. Brabender Instruments, Inc. of South Hackensack, N.J. Other suitable apparatus will be apparent to those of ordinary skill in the art.

A further aspect of the invention is related to the preparation of melt extruded multiparticulates as set forth above in a manner which controls the amount of air included in tile extruded product. By controlling the amount of air included in the extrudate, it has been surprisingly found that the release rate of the therapeutically active agent from the, e.g., multiparticulate extrudate, can be altered significantly.

Yet another aspect of the invention, the melt extruded product is prepared in a manner which substantially excludes air during the extrusion phase of the process. This may be accomplished, for example, by using a Leistritz extruder having a vacuum attachment.

### General Pellet Manufacturing Procedure

The following technique was used to manufacture the extrudate and multiparticulates for Examples 1-4:

Blend the required amount of drug, hydrophobic material and binder along with any additional excipients.

Charge a powder feeder with proper amount of drug/excipient blend.

Set temperatures of extruder heating zones to the required temperature, depending on the formulation. Typically, the temperature should be set at about 83° C. Wait until the corresponding heating zones reach steady temperatures. Set the extruder screw rotation speed to 20 rpm. Start the feeder, the conveyor and the pelletizer. After the excipients are melted and the drug is embedded in the molten mixture, the resultant viscous mass is extruded as spaghetti-like strands. The diameter of the extruder aperture can be adjusted to vary the thickness of the resulting strand.

Set the conveyor belt speed to an appropriate speed (e.g., 3-100 ft/min). Allow the extruded semisolid strand(s) to be congealed and/or hardened while transported to the pelletizer on the conveyor belt. Additional cooling devices may be needed to ensure proper congealing. (The conveyor belt may not be needed to cool the strand, if the material congeals rapidly enough.)

Set the roller knife to an appropriate speed (e.g., to 3-100 ft/min and 100-800 rpm). Cut the congealed strands to desired size (e.g., 3-5 mm in diameter, 0.3-5 mm in length).

Collect the pellet product.

Fill a desired weight of pellets into hard gelatin capsules to obtain an appropriate doses of the drug, e.g oxycodone.

### Dissolution Method

The following dissolution method may be used to determine the dissolution profile of a sustained-release oxycodone formulaiton prepared in accordance with the present invention:

(USP II Paddle at 100 rpm at 37° C.)

Media—1st hour in 700 ml simulated gastric fluid (SGF), pH 1.2 without enzyme thereafter, 900 ml simulated intestinal fluid (SIF), pH 7.5 without enzyme, using HPLC procedures for assay.

The following examples illustrate various aspects of the present invention. They are not to be construed to limit the claims in any manner whatsoever.

### EXAMPLE 1

A controlled release oxycodone formulation which dissolves preferentially in high pH intestinlal fluid rather than in the low pH gastric fluid was prepared according to the formula provided in Table 1 below:

TABLE 1

| | | Percentage |
|---|---|---|
| Ingredients | Amt (mg)/Capsule | in Formula |
| Oxycodone HCl | 20 | 25 |
| Eudragit RSPO | 39 | 48.75 |
| Eudragit L-100 | 3 | 3.75 |
| Stearic Acid | 18 | 22.5 |
| Total | 80 | 100 |

The formulation of Example 1 was prepared as follows:

Pellet Manufacture

a. Extruder system description—The twin screw extruder is consisted of a pair of counterrotating screws and a barrel block equipped with heating/cooling zones. The extrudate is delivered to a pelletizer through a conveyor belt and cut into pellets of the desirable size.

b. Manufacturing procedure—

1. Blend the drug and all the excipients in a proper mixer.

2. Place the mixture in a powder feeder.

3. Set temperatures of the extruder heating zones to approximately 83° C.

4. Set the extruder screw rotation speed to 20 rpm.

5. Start the feeder, the conveyor and the pelletizer.

6. After the excipients are melted and the drug embedded in the molten mixture, the viscous mass is extruded as spaghetti-like strands.

7. The extrudate is congealed and hardened while being delivered to the pelletizer on the conveyor belt.

8. The roller knife of the pelletizer cuts the strands into pellets of 1.5 mm in diameter and 1.5 mm in length.

Encapsulation

After the pellets were manufactured, 80 mg of pellets are encapsulated in size #2 hard gelatin capsules, rendering capsules containing 20 mg of oxycodone HCl. These capsules were then tested using the following dissolution methodology:

1. Apparatus—USP Type II (paddle), 100 rpm at 37° C.

2. Media—Either 900 ml simulated gastric fluid (SGF), pH 1.2 without enzyme; or 900 ml simulated intestinal fluid (SIF), pH without enzyme.

3. Analytical method—High Performance liquid chromatography (HPLC)

The dissolution results are set forth in Table 2 below:

TABLE 2

| Time (hr) | 1 | 2 | 4 | 8 | 12 | 18 | 24 |
|---|---|---|---|---|---|---|---|
| Mean % dissolved (SGF) | 13 | 20 | 29 | 41 | 51 | 62 | 71 |
| Mean % dissolved (SIF) | 14 | 21 | 31 | 44 | 57 | 68 | 80 |

### EXAMPLE 2

A bioavailability study of oxycodone controlled release capsules of Example 1 was conducted in 10 normal male volunteers. These capsules were administered either with or without food. The study was conducted in a single dose crossover design. Blood samples were taken periodically and assayed for oxycodone concentrations using gas chromatography with mass detection (GC/MS). The plasma oxycodone concentration versus time curves are shown in FIG. 1.

From the data, the following pharmacokinetic parameters were calculated as set forth in Table 3 below:

TABLE 3

| | AUC, | | |
|---|---|---|---|
| Treatment | n. hr/ml | Cmax, n/ml | Tmax, hr |
| Example 1, fasted | 207 | 9.7 | 5.3 |
| Example 1, fed | 261 | 14.8 | 6.4 |

From the data provided in Table 3, it can be seen that the formulation of Example 1 has a fed/fast effect as defined herein. One skilled in the art would not necessarily be surprised by this outcome. It merely represents a common approach to avoiding a fed/fast effect which in this case didn't work.

### EXAMPLE 3

An oxycodone HCl controlled release tablet which would dissolve preferentially in a lower pH, the following formula is used:

TABLE 4

| Ingredients | Amt (mg)/Tablet | Percentage in Formula |
|---|---|---|
| Oxycodone HCl | 40 | 30.8 |
| Eudragit RS30D (solid) | 14 | 10.8 |
| Spray Dried Lactose | 35.25 | 27.1 |
| PVP | 5 | 3.9 |
| Triacetin | 2 | 1.5 |
| Stearyl Alcohol | 25 | 19.2 |
| Talc | 2.5 | 1.9 |
| Magnesium Stearate | 1.25 | 0.9 |
| Film Coat | 5 | 3.9 |
| Total | 130 | 100 |

Total Manufacture

1. Mix Eudragit RS30D (suspension) and Triacetin for 5 minutes.

2. Place spray dried lactose, oxycodone HCl, PVP, in a fluid bed drier.

3. Spray the suspension onto the powders under fluidization.

4. Pass the granulation through a Comil to reduce lumps.

5. Melt stearyl alcohol at 70° C.

6. Incorporate the molten stearyl alcohol into the dry granulation in a Collete Mixer.

7. Transfer the waxed granulation to a cooling tray and allow the granulation to congeal.

8. Pass the granulation through a Comil.

9. Mix the waxed granulation with talc and magnesium stearate in a Collete Mixer.

10. Compress the lubricated granulation into tablets using a rotary tablet press.

11. Film coat the tablets.

These tablets were then tested using the following dissolution methodology described in Example 1.

The above tablets were found to have the following dissolution results:

TABLE 5

| Time (hr) | 1 | 2 | 4 | 8 | 12 |
|---|---|---|---|---|---|
| Mean % dissolved SGF | 39 | 53 | 70 | 90 | 99 |
| Mean % dissolved SIF | 35 | 48 | 65 | 83 | 93 |

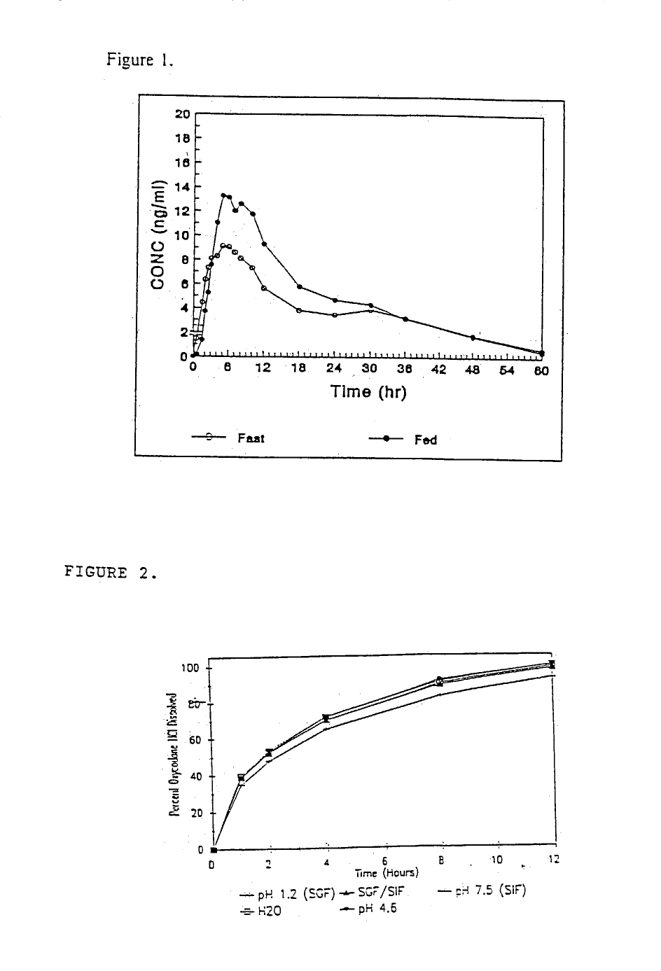

Further information concerning the effect of different dissolution media pH on release rate of the oxycodone from the formulation of Example 3 is set forth in FIG. 2. These results set forth above clearly indicate that the dissolution profile of the tablets of Example 3 are insensitive to variations in dissolution media pH in the range of 1.2 to 6.8. The rate of drug substance release does, however, begin to decrease as the pH of the dissolution medium becomes basic. The SGF/SIF pH change curve is characteristic of profiles generated in media whose pH is less than 7, most likely because the initial time point is generated in an acidic medium. Following the change in pH to 7.5, the dissolution curve is parallel to that generated in the basic medium, but off-set by the significantly higher release in the first hour at acidic pH. Nevertheless, the amount of drug substance released from the drug product at every time point using SGF/SIF is comparable to that obtained in acidic media only.

### EXAMPLE 4

### Bioavailability of Example 3 Tablets

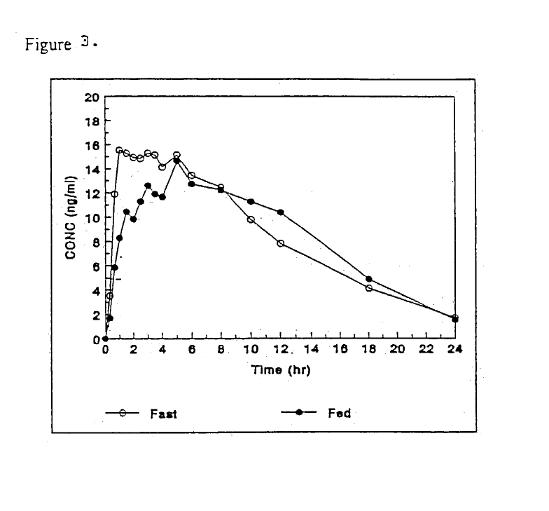

A bioavailability study of oxycodone controlled release tablets of Example 3 was conducted in 25 normal volunteers. These tablets were administered either with or without food. The study was conducted in a single dose, randomized crossover design. Blood samples were taken periodically and assayed for oxycodone concentrations using gas chromatography with mass detection (GC/MS). The plasma oxycodone concentration versus time curves are shown in FIG. 3.

From the data, the following pharmacokinetic parameters were calculated.

TABLE 6

| Treatment | AUC, ng. hr/ml | Cmax, ng/ml | Tmax, hr |
|---|---|---|---|
| Example 3, fasted | 422 | 39.3 | 3.1 |
| Example 3, fed | 416 | 35.3 | 4.8 |

Surprisingly, it was found that the controlled release oxycodone HCl preparation, which dissolved preferentially in low pH, does not show substantial food effect. From the Cmax data, it can be seen that there is no significant change in blood oxycodone levels when the drug was taken with food than without food (35.3/39.3=0.09). From the AUC (area under the curve) data, it appears that the amount of drug absorbed with or without food is similar (416/422=0.986).

Many other variations of the present invention will be apparant to those skilled in the art and are meant to be within the scope of the claims appended hereto.

What is claimed is:

1. An oral solid sustained-release oral dosage form containing oxycodone which is bioavailable and does not exhibit a fed/fast effect, comprising
an analgesically effective amount of oxycodone or a salt thereof;
an effective amount of a sustained release carrier which preferentially causes said oxycodone to be released faster in simulated gastric fluid than in simulated intestinal fluid to provide analgesia for a time period from about 8 to about 24 hours when said dosage form is orally administered to a patient; and optional pharmaceutical excipients.

2. A dosage form according to claim 1 wherein said therapeutically effective amount of oxycodone is from about 2 mg to about 500 mg, calculated based on the hydrochloride salt.

3. A dosage form according to composition of claim 1, wherein said susteined-release carrier comprises an acrylic resin.

4. A dosage form according to claim 1 which provides effective blood levels of oxycodone when administered to human patients for about 12 hours for about 24 hours.

5. A method for the preparation of an oral sustained release oxycodone formulation, the improvement comprising utilizing a sustained release carrier which preferentially releases oxycodone or a pharmaceutically acceptable salt thereof faster in simulated gastric fluid than in simulated intestinal fluid, such that the formulation is bioavailable and does not exhibit a fed/fast effect.

6. A method of treating patients having moderate to severe pain, comprising administering an oral sustained-release formulation comprising effective amount of oxycodone or a pharmaceutically acceptable salt thereof in a sustained release carrier which preferentially releases oxycodone or a pharmaceutically acceptable salt thereof faster in simulated gastric fluid than in simulated intestinal fluid, such that the formulation is bioavailable and does not exhibit a fed/fast effect.

7. A method of preparing an oral sustained-release oxycodone formulation which is bioavailable and which does not exhibit a fed/fast effect, comprising,
preparing a solid dosage form comprising an effective amount of oxycodone or a pharmaceutically acceptable salt thereof in a sustained release carrier which preferentially releases oxycodone or a pharmaceutically acceptable salt thereof faster in simulated gastric fluid than in simulated intestinal fluid, and optional pharmaceutical excipients, such that the formulation is bioavailable and does not exhibit a fed/fast effect.
